(19) United States
(12) Patent Application Publication
(10) Pub. No.: US 2018/0264096 A1
(43) Pub. Date: Sep. 20, 2018

(54) Prophylactic Vaccine to Tumor Angiogenesis

(71) Applicant: Batu Biologics, Inc., San Diego, CA (US)

(72) Inventors: Samuel C. Wagner, San Diego, CA (US); Thomas E. Ichim, San Diego, CA (US)

(21) Appl. No.: 15/924,629

(22) Filed: Mar. 19, 2018

Related U.S. Application Data
(60) Provisional application No. 62/473,942, filed on Mar. 20, 2017.

(51) Int. Cl. A61K 39/00; A61P 35/00

(52) U.S. Cl. CPC A61K 39/0011; A61K 2039/585; A61P 35/00

(57) ABSTRACT

Disclosed are compositions of matter, protocols, and treatment means for inducing an enhanced immunity targeting tumor endothelium in order to prophylactically protect subjects from development of neoplasia. In one embodiment, the invention provides placental endothelial cells that are tissue culture expanded under proliferative conditions to resemble the tumor endothelial driven angiogenesis. The cells are subsequently treated with interferon gamma to increase immunogenicity and utilized as a prophylactic vaccine. Antibody and cell mediated immunity towards tumor endothelial associated antigens is quantified with the aim of establishing protective immunity which inhibits or blocks development of angiogenesis-dependent neoplasia.

### CROSS-REFERENCE TO RELATED APPLICATIONS

This patent application takes priority from Provisional Patent Application No. 62/473,942, titled Prophylactic Vaccine to Tumor Angiogenesis, filed on Mar. 20, 2017, the contents of which are expressly incorporated herein by this reference as though set forth in their entirety and to which priority is claimed.

### FIELD OF THE INVENTION

The invention pertains to the field of prophylactic tumor vaccines, more specifically, the invention deals with generation of therapeutic cancer vaccines that specifically target tumor vasculature, thus choking off tumor blood supply. The invention further pertains to anti-angiogenesis prophylactically before disease ensues.

### BACKGROUND

Vaccines are an attractive means for preventing, slowing, or prohibiting the development of recurrent disease due to their ease of administration, and because of their high rate of success observed for infectious diseases. The basic concept of constructing cancer vaccines is straightforward in theory. The development of effective cancer vaccines for solid tumors in practice, however, has met with limited success. For example, one group attempting to administer a peptide vaccine directed against metastatic melanoma observed an objective response rate of only 2.6%. There are many potential explanations for this low success rate. For example, even if an antigen is specifically associated with a particular type of tumor cell, the tumor cells may express only low levels of the antigen, or it may be located in a cryptic site or otherwise shielded from immune detection. In addition, tumors often change, their antigenic profile by shedding antigens as they develop. Also contributing to the low success rate is the fact that tumor cells may express very low levels of MHC proteins and other co-stimulatory proteins necessary to generate an immune response.

The immune system effectively delays the onset and reduces the incidence of tumors, as illustrated by the enormous increase in neoplastic growth, spontaneous and carcinogen induced, observed in severely immunodepressed mice. Unfortunately, the existence of tumors in mice and humans clearly shows that spontaneous immune responses are not sufficient for a complete prevention of carcinogenesis. Conversely, it has been shown that a prophylactic activation of the immune system with vaccines and cytokines could result in a significant reduction in tumorigenesis. For example, tumors were induced in BALB/c mice by s.c. injection of 3-methylcholanthrene. Delayed tumor appearance and reduced incidence were observed in mice receiving 100 ng of systemic IL-12 five days a week for 18 weeks (3 weeks on and 1 week off) during tumor latency. Secondary IFN-γ, IL-10, and tumor necrosis factor-α were evident in their sera. A high production of IFN-γ by CD8 T cells and a Th2→Th1 or Th0 shift in the cytokine secretion profile of CD4 T cells were also noted.

The effectiveness of tumor immunoprevention has been clearly shown for carcinogen-induced tumors and for spontaneous tumors originating in transgenic mouse models. A wide array of different immunologic strategies was used to prevent carcinomas in HER-2/neu transgenic mice, including passive administration of anti-HER-2/neu antibodies, modulators of the immune response like interleukin 12 (IL-12), α-galactosylceramide, or bacterial CpG sequences, and various HER-2/neu-specific vaccines based on whole cells, DNA, peptides, protein, or heat shock proteins. An almost complete immunoprevention of mammary carcinogenesis was obtained with a cell vaccine combining different immunologic stimuli. All of these have been reviewed in Croci, S., et al., Immunological prevention of a multigene cancer syndrome. Cancer Res, 2004. 64(22): p. 8428-34.

Unfortunately, clinical translation has been hampered by toxicity or potential toxicity of immunogens, as well as lack of efficacy. By utilizing naturally occurring placental endothelial cells as an immunogen, and targeting malignant blood vessels, instead of the tumor itself, the invention provides a new, useful and non-obvious way to induce successful protection against cancer.

### DESCRIPTION OF THE INVENTION

“Marker” and “Biomarker” are used interchangeably to refer to a gene expression product that is differentially present in samples taken from two different subjects, e.g., from a test subject or patient having (a risk of developing) an ischemic event, compared to a comparable sample taken from a control subject (e.g., a subject not having (a risk of developing) an ischemic event; a normal or healthy subject). Alternatively, the terms refer to a gene expression product that is differentially present in a population of cells relative to another population of cells.

The phrase “differentially present” refers to differences in the quantity or frequency (incidence of occurrence) of a marker present in a sample taken from a test subject as compared to a control subject. For example, a marker can be a gene expression product that is present at an elevated level or at a decreased level in blood samples of a risk subjects compared to samples from control subjects. Alternatively, a marker can be a gene expression product that is detected at a higher frequency or at a lower frequency in samples of blood from risk subjects compared to samples from control subjects.

A gene expression product is “differentially present” between two samples if the amount of the gene expression product in one sample is statistically significantly different from the amount of the gene expression product in the other sample. For example, a gene expression product is differentially present between two samples if it is present at least about 120%, at least about 130%, at least about 150%, at least about 180%, at least about 200%, at least about 300%, at least about 500%, at least about 700%, at least about 900%, or at least about 1000% greater than it is present in the other sample, or if it is detectable in one sample and not detectable in the other.

As used herein, the terms “antibody” and “antibodies” refer to monoclonal antibodies, multispecific antibodies, synthetic antibodies, human antibodies, humanized antibodies, chimeric antibodies, single-chain Fvs (scFv), single chain antibodies, Fab fragments, F(ab′) fragments, disulfide-linked Fvs (sdFv), and anti-idiotypic (anti-Id) antibodies (including, e.g., anti-Id antibodies to antibodies of the invention), and epitope-binding fragments of any of the above. In particular, antibodies of the present invention include immunoglobulin molecules and immunologically active portions of immunoglobulin molecules, i.e., molecules that contain an antigen binding site that immunospecifically binds to a polypeptide antigen encoded by a gene comprised in the genomic regions or affected by genetic transformations in the genomic regions listed in Table 1. The immunoglobulin molecules of the invention can be of any type (e.g., IgG, IgE, IgM, IgD, IgA and IgY), class (e.g., IgG₁, IgG₂, IgG₃, IgG₄, IgA₁ and IgA₂) or subclass of immunoglobulin molecule.

“Immunoassay” is an assay that uses an antibody to specifically bind an antigen (e.g., a marker). The immunoassay is characterized by the use of specific binding properties of a particular antibody to isolate, target, and/or quantify the antigen. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Antibodies, A Laboratory Manual (1988), for a description of immunoassay formats and conditions that can be used to determine specific immunoreactivity). Typically a specific or selective reaction will be at least twice background signal or noise and more typically more than 10 to 100 times background.

The phrase “specifically (or selectively) binds” when referring to an antibody, or “specifically (or selectively) immunoreactive with”, when referring to a protein or peptide, refers to a binding reaction that is determinative of the presence of the protein in a heterogeneous population of proteins and other biologics. Thus, under designated immunoassay conditions, the specified antibodies bind to a particular protein at least two times the background and do not substantially bind in a significant amount to other proteins present in the sample. Specific binding to an antibody under such conditions may require an antibody that is selected for its specificity for a particular protein.

The terms “affecting the expression” and “modulating the expression” of a protein or gene, as used herein, should be understood as regulating, controlling, blocking, inhibiting, stimulating, enhancing, activating, mimicking, bypassing, correcting, removing, and/or substituting the expression, in more general terms, intervening in the expression, for instance by affecting the expression of a gene encoding that protein.

In one embodiment, EPCs refer to endothelial colony-forming cells (ECFCs) and their progenitor cell capacities were characterized as described (Wu, Y et al., J Thromb Haemost, 2010; 8:185-193; Wang, H et al., Circulation research, 2004; 94:843 and Stellos, K et al., Eur Heart J., 2009; 30:584-593). Briefly, human blood was collected from healthy volunteer donors. All volunteers had no risk factors of CVD including hypertension, diabetes, smoking, positive family history of premature CVD and hypercholesterolemia, and were all free of wounds, ulcers, retinopathy, recent surgery, inflammatory, malignant diseases, and medications that may influence EPC kinetics. After dilution with HBSS (1:1), blood was overlaid onto Histopaque 1077 (Sigma-Aldrich Co. LLC, St. Louis, Mo.) in the ratio of 1:1 and centrifuged at 740 g for 30 minutes at room temperature. Buffy coat MNCs were collected and centrifuged at 700 g for 10 minutes at room temperature. MNCs were cultured in collagen type I (BD Bioscience, San Diego) (50 m/ml)-coated dishes with EBM2 basal medium (Lonza Inc., Allendale, N.J.) plus standard EGM-2 SingleQuotes (Lonza Inc., Allendale, N.J.) that includes 2% fetal bovine serum (FBS), EGF (20 ng/ml), hydrocortisone (1 μg/ml), bovine brain extract (12 μg/ml), gentamycin (50 m/ml), amphotericin B (50 ng/ml), and epidermal growth factor (10 ng/ml). Colonies appeared between 5 and 22 days of culture were identified as a well-circumscribed monolayer of cobblestone-appearing cells. ECFCs with endothelial lineage markers expression, robust proliferative potential, colony-forming, and vessel-forming activity in vitro are defined as EPCs as described (Wang, H et al., Circulation research, 2004; 94:843 and Stellos, K et al., Eur Heart J., 2009; 30:584-593). Passage 4 to 6 EPCs were used for experiments. For a brief characterization, endothelial phagocytosis function was confirmed by incubating EPC in 4-well chamber slide with 1, 1-dioctadecyl-3, 3, 3, 3-tetramethylindocarbocyanine (DiI)-labeled acetylated low-density lipoprotein (acLDL) (Biomedical Technologies, Inc., Stoughton, Mass.) (5 m/ml) at 37° C. for 1 h, washed 3 times for 15 min in PBS, and then fixed with 2% paraformaldehyde for 10 min. Cells were then incubated with FITC conjugated UEA-1 (Ulex europaeus agglutinin) (10 m/ml) (Sigma-Aldrich Corporation, St. Louis, Mo.) for 1 h at room temperature, which is capable of binding with glycoproteins on the cell membrane to allow visualization of the entire cell. Cell integrity was examined by nuclear staining with DAPI (100 ng/ml). After staining, cells are imaged with high-power fields under an inverted fluorescent microscope (Axiovert 200, Carl Zeiss, Thornwood, N.Y.) at 200× magnification and quantified using Image J software.

In one embodiment, the invention provides administration of endothelial progenitor cells derived from pluripotent stem cells as a means of inducing prophylactic cancer immunity. Means of utilizing progenitor cells for generation of immune responses, both humoral and cellular are described in the art and incorporated by reference. For example, Zheng et al described the efficacy of two stem cell-based vaccines in the prophylaxis and treatment of subcutaneous hepatic tumors transplanted into mice. C57BL/6j mice were vaccinated weekly with either hepatic stem cells (HSCs) or embryonic stem cells (ESCs) for three weeks, followed by a subcutaneous challenge with Hepa 1-6 cells at one week (group 1) or four weeks (group 2) after vaccination. No tumor formation was observed in HSC-vaccinated mice when challenged within one week after vaccination (group 1), but tumors formed in 10% of mice in the ESC-vaccinated group and in 60% of mice in the unvaccinated group. When the long-term memory response was examined (group 2), only 10% of HSC-vaccinated mice and 20% of ESC-vaccinated mice developed macroscopic hepatocarcinomas compared to 60% of the unvaccinated mice. Besides their function as prophylactic vaccines, administration of either HSC or ESC could be a potential treatment for cancer. In mice with subcutaneous hepatocarcinomas, complete clearance of tumor burden was observed in 80% of mice receiving HSC vaccination, but 40% of ESC-vaccinated mice presented with tumors that did not increase in size over time.

In one embodiment of the invention, the practitioner leverages the theory that oncogenesis occurs from nests of embryonal stem cells, present in normal tissues and stimulated to grow by some kind of irritation or chemical exposure. Supporting this theory, there is now evidence that mutated, tissue-specific stem cells act as self renewing “cancer initiating cells”, responsible for the initiation of many malignancies. In indirect support of this idea, there is abundant evidence that most solid tumor types express embryonic antigens to varying degrees For example, the ‘carcinoembryonic antigens’, first described in the mid-1960s, represent antigens shared by embryos and tumors of the digestive tract. Accordingly, in one aspect of the invention prophylactic immunization is performed using placental endothelial progenitor cells, or other progenitor cells that possess tumor antigens that are shared with fetal and/or placental antigens. For example, there is a report in which antisera raised in rabbits against an emulsified whole human embryo (6-7 week)—adsorbed against adult human tissues—recognized a variety of human tumor types including skin, bronchial, renal, colonic, hepatic, lung and breast. These observations support the concept that animals or humans immunized against embryonic material might be capable of recognizing and destroying neoplastic cells. The materials and methods used for induction of immunity may be leveraged in the case of the present invention for induction of prophylactic immunity against tumor blood vessels.

In one embodiment of the invention, stem cells are used as a means of inducing prophylactic immunity, with the stem cells being differentiated in whole or in part towards the endothelial cell lineage. In one embodiment xenongeneic stem cells are used. For example, the murine embryonic stem cell (ESC) line, ES-D3 (ATCC CRL-11632), derived from 129/Sv mice (expressing MHC class II I-E). ESC may be cultured under 5% CO2 in Dulbecco's modified eagle's medium (DMEM) supplemented with 15% ES Cell Qualified fetal bovine serum, 50 U/ml penicillin, 50 μg/ml streptomycin, 0.1 mM non-essential amino acids, 0.1 mM f3-mercaptoethanol and 2 mM L-glutamine (all from GIBCO, Invitrogen Corporation, Grand Island, N.Y.) under standard conditions. No feeder layer may be used if leukemia inhibitory factor (Chemicon, Temecula, Calif.) is added at a concentration of 80 units/ml (500 pM) to prevent differentiation of the ESC during culture. ESC are periodically evaluated using anti-SSEA-1 (MC-480, Developmental Studies Hybridoma Bank, Iowa City, Iowa) and with BD Stemflow™ human and mouse pluripotent stem cell analysis kit (BD Biosciences, San Jose, Calif.) to ensure retention of an undifferentiated state. For generation of vaccines, stem cells are removed from the plate with enzyme-free cell dissociation solution (Specialty Media, Phillipsburg, N.J.), washed twice in sterile Hank's buffered salt solution (HBSS) and suspended in HBSS at a concentration of 10×10⁶/ml. The cells were injected subcutaneously (s.c), 1×10⁶ per inoculation. Amount of injection differs on the patient characteristics and immune response desired. Murine fibroblasts expressing GM-CSF (1×10⁶ per inoculation) may be co-administered with the ESC (ESC/STO-GM vaccine). STO fibroblast cell line (ATCC # CRL-1503) are infected in culture with a replication-defective retrovirus expressing murine GM-CSF and maintained and processed under the same conditions as the ESC. GM-CSF production by these cells is ensured by ELISA measurements on cell supernatants (R & D Systems, Minneapolis, Minn.). ESC and STO-GM cells may be irradiated (15 Gy) before immunization. Other means of immunizing using stem cells are described in the literature that may be applied to stem cells differentiated into the endothelial lineage. These include a study in which researchers investigated whether vaccination with defined human embryonic stem cells (hESCs) or induced pluripotent stem (iPS) cells was effective against a colon carcinoma. It was demonstrated that vaccination of mice with hESC line H9 generated consistent cellular and humoral immune responses against CT26 colon carcinoma. Protection correlated strongly with the expansion of tumor-responsive and interferon-gamma-producing cells and the profound loss of CD11b(+)Gr-1(+) myeloid-derived suppressor cells in the spleen. No evidence of autoimmunity was observed. They also compared the immunogenicity against colon cancer between a hESC line CT2 and an iPS cell line TZ1 that were generated in the same stem cell facility. It was found that the iPS cell line was inferior to the hESC line in conferring tumor protection. Others have described techniques for immunization with stem cells, which are incorporated by reference. One example of an antigen that is found both on embryonic stem cells, on placental stem cells, and on cancer stem cells is SSEA3, another one is SOX2.

In one specific embodiment, the invention teaches generation of antibody and cellular responses to antigens found on cancer stem cells, and cancer stem cell associated blood vessels. In particular, the invention teaches that immunization with placental endothelial cells that have been cultured in conditions resembling the tumor microenvironment induce generation of immune responses that cross react with cancer stem cells, and/or cancer stem cell associated blood vessels. Specifically, cancer stem cells (CSCs), are rare cells with the ability of self-renewal and tumor initiation, are closely related to cancer progression and specific targets for effective therapy and early diagnosis. CSCs have been identified and characterized by protein markers, as in the case of breast CSCs (BCSCs) which were first discovered in 2003 by Al-Hajj et al. who demonstrated that breast cancer cells with CD44⁺CD24^{-/lo} expression have higher level of tumorigenicity than others and can form tumor in animals with ˜100 of such cells. In addition, other proteins such as ALDH-1, CD133, CD326 (ESA), CD201 (PROCR), and their combinations, are also reported as BCSCs biomarkers. However, the BCSCs obtained from the enrichment process based on these markers still contain a large number of noncancer stem cells, and study of such cells would provide nonspecific characteristics of CSCs. Another type of antigens that are targeted by the use of the current invention are glycolipids. Glycolipids are known to be altered during cancer development and embryongenesis. In one study it was shown that the globo-series glycans SSEA-3 (Gb5), SSEA-4 (sialyl-Gb5), and globo-H (fucosyl-Gb5) are found exclusively on the cell surface of many cancers, including breast cancer and BCSCs, as well as in embryonic tissue. It has also been reported that BCSCs carrying either ESA^{hi}PROCR^{hi} or CD44⁺CD24^{-/lo} showed high expression of these globo-series epitopes. SSEA-3 is synthesized from Gb4 by β3GalT5, and globo-H and SSEA-4 are synthesized from SSEA-3 by fucosyltransferases 1 and 2 (FUT1, FUT2) and ST3 β-galactoside α-2,3-sialyltransferase 2 (ST3Gal2), respectively. Accordingly in some embodiments placental endothelial cells are modified to express tumor antigens in order to generate prophylactic immunity not only toward tumor blood vessels but also towards tumor stem cells themselves.

The invention provides means of utilizing endothelial progenitor cells and products derived from the endothelial progenitor cells as a prophylactic cancer vaccine which selectively induces immunity towards tumor vasculature and not healthy, non-malignant, vasculature. In one embodiment the invention teaches the utilization of culture conditions which mimic the tumor microenvironment as a means of creating a cellular population that resembles tumor endothelial cells. Culture conditions include the growth of endothelial progenitor cells in acidic conditions which resemble the tumor microenvironment. Numerous papers have characterized the acidic conditions in the tumor microenvironment and are incorporated by reference. See Damaghi, M. and R. Gillies, Phenotypic changes of acid adapted cancer cells push them toward aggressiveness in their evolution in the tumor microenvironment. Cell Cycle, 2016: p. 0; Lobo, R. C., et al., Glucose Uptake and Intracellular pH in a Mouse Model of Ductal Carcinoma In situ (DCIS) Suggests Metabolic Heterogeneity. Front Cell Dev Biol, 2016. 4: p. 93; An, S., et al., Amino Acid Metabolism Abnormity and Microenvironment Variation Mediated Targeting and Controlled Glioma Chemotherapy. Small, 2016; Avnet, S., et al., Altered pH gradient at the plasma membrane of osteosarcoma cells is a key mechanism of drug resistance. Oncotarget, 2016; Huang, S., et al., Acidic extracellular pH promotes prostate cancer bone metastasis by enhancing PC-3 stem cell characteristics, cell invasiveness and VEGF-induced vasculogenesis of BM-EPCs. Oncol Rep, 2016. 36(4): p. 2025-32; Carnero, A. and M. Lleonart, The hypoxic microenvironment: A determinant of cancer stem cell evolution. Bioessays, 2016. 38 Suppl 1: p. S65-74; Pellegrini, P., et al., Tumor acidosis enhances cytotoxic effects and autophagy inhibition by salinomycin on cancer cell lines and cancer stem cells. Oncotarget, 2016. 7(24): p. 35703-35723; Bohme, I. and A. K. Bosserhoff, Acidic tumor microenvironment in human melanoma. Pigment Cell Melanoma Res, 2016. 29(5): p. 508-23; Gentric, G., V. Mieulet, and F. Mechta-Grigoriou, Heterogeneity in Cancer Metabolism: New Concepts in an Old Field. Antioxid Redox Signal, 2016; Ge, Y., et al., Preferential extension of short telomeres induced by low extracellular pH. Nucleic Acids Res, 2016. 44(17): p. 8086-96; Quail, D. F., et al., The tumor microenvironment underlies acquired resistance to CSF-1R inhibition in gliomas. Science, 2016. 352(6288): p. aad3018; Li, X., et al., The altered glucose metabolism in tumor and a tumor acidic microenvironment associated with extracellular matrix metalloproteinase inducer and monocarboxylate transporters. Oncotarget, 2016. 7(17): p. 23141-55; Zhao, C., et al., Tumor Acidity-Induced Sheddable Polyethylenimine-Poly(trimethylene carbonate)/DNA/Polyethylene Glycol-2,3-Dimethylmaleicanhydride Ternary Complex for Efficient and Safe Gene Delivery. ACS Appl Mater Interfaces, 2016. 8(10): p. 6400-10; and Riemann, A., et al., Acidosis Promotes Metastasis Formation by Enhancing Tumor Cell Motility. Adv Exp Med Biol, 2016. 876: p. 215-20.

Interestingly, tumor acidic conditions are believed to be associated with resistance to immunotherapy. In a recent study it was shown that an acidic pH environment blocked T-cell activation and limited glycolysis in vitro. IFNγ release blocked by acidic pH did not occur at the level of steady-state mRNA, implying that the effect of acidity was posttranslational. Acidification did not affect cytoplasmic pH, suggesting that signals transduced by external acidity were likely mediated by specific acid-sensing receptors, four of which are expressed by T cells. Notably, neutralizing tumor acidity with bicarbonate monotherapy impaired the growth of some cancer types in mice where it was associated with increased T-cell infiltration. Furthermore, combining bicarbonate therapy with anti-CTLA-4, anti-PD1, or adoptive T-cell transfer improved antitumor responses in multiple models, including cures in some subjects [40]. In one embodiment of the invention, endothelial progenitor cells, or products thereof, are cultured under conditions in GCN2 kinase is activated, the conditions include culture in the presence of uncharged tRNA, tryptophan deprivation, arginine deprivation, asparagine deprivation, and glutamine deprivation.

The potential of using the tumor vasculature as a target is enticing, however previous studies have not utilized polyvalent antigenic entities, or in the cases where they have, such as in cellular vaccines, the cells where either not made to be immunogenic, nor are the cells grown under conditions which induce replicate the tumor microenvironment. The following examples are provided to allow the practitioner of the invention to ascertain various immunization regimens, adjuvants, and combinations. The invention teaches means of “focusing” an immune response subsequent to immunization with a polyvalent cancer vaccine targeting tumor associated blood vessels. In one embodiment, patients suffering from cancer are immunized with ValloVax, or a vaccine composition similar to tumor endothelial cells. Active immunization against tumor endothelium by vaccinating against proliferating endothelium or markers found on tumor endothelium has provided promising preclinical data. Specifically, in animal models it has been reported that immunization to antigens specifically found on tumor vasculature can lead to tumor regression. Studies have been reported using the following antigens: survivin, endosialin, and xenogeneic FGF2R, VEGF, VEGF-R2, MMP-2, and endoglin. Human trials have been conducted utilizing human umbilical vein endothelial (HUVEC) cells as tumor antigens, with responses being reported in patients. In one report describing a 17-patient trial, Tanaka et al demonstrated that HUVEC vaccine therapy significantly prolonged tumor doubling time and inhibited tumor growth in patients with recurrent glioblastoma, inducing both cellular and humoral responses against the tumor vasculature without any adverse events or noticeable toxicities.

The invention provides the use of tissue or circulating EPC as a substrate for transformation into an immunogenic cell population resembling tumor associated endothelial cells. The EPC is an undifferentiated cell that can be induced to proliferate using the methods of the present invention. The EPC is capable of self-maintenance, such that with each cell division, at least one daughter cell will also be an EPC cell. EPCs are capable of being expanded 100, 250, 500, 1000, 2000, 3000, 4000, 5000 or more fold. Phenotyping of EPCs reveals that these cells express the committed hematopoietic marker CD45. Additionally, an EPC is immunoreactive for VEGFR-2. The EPC is a multipotent progenitor cell. By multipotent progenitor cell is meant that the cell is capable of differentiating into more than one cell type. For example, the cell is capable of differentiating into an endothelial cell or a smooth muscle cell. Vascular endothelial growth factor (VEGF) acts through specific tyrosine kinase receptors that includes VEGFR-1 (flt-1) and VEGFR-2 (flk-1/KDR) and VEGFR-3/Flt-4 which convey signals that are essential for embryonic angiogenesis, cancer angiognesis and hematopoiesis. While VEGF binds to all three receptors, most biological functions are mediated via VEGFR-2 and the role of VEGFR-1 is currently unknown. VEGFR3/Flt4 signaling is known to be important for the development of lymphatic endothelial cells and VEGFR3 signaling may confer lymphatic endothelial-like phenotypes to endothelial cells. VEGFRs relay signals for processes essential in stimulation of vessel growth, vasorelaxation, induction of vascular permeability, endothelial cell migration, proliferation and survival. Endothelial cells express all different VEGF-Rs. During embryogenesis, it has been reported that a single progenitor cell, the hemangioblast can give rise to both the hematopoietic and vascular systems. In the process of tumor angiogenesis, VEGF plays a fundamental role in promoting malignant and leaky angiogenesis.

It is known that VEGF is stimulated in part by hypoxia, and by activation of SDF-1 through HIF-1 alpha. In one embodiment, endothelial progenitor cells are treated with a combination of VEGF and other tumor associated factors. In order to induce the generation of endothelial cells the resemble tumor endothelial cells, culture under conditions that stimulate HIF-1 alpha are used. In one embodiment the invention discloses means of modifying through culture endothelial cells. One type of culture condition involves growth of cells under hypoxia. Numerous means of culturing cells in hypoxia are known in the art and are described in the following references. Other means of inducing cellular signaling mimicking hypoxia include treatment with tissue factor, or tissue factor activating compounds. Other approaches to activating these pathways include treatment with LRG-1, culture with macrophages, treatment with CCLS, culture with lactic acid alone or in the presence of monocytes, culture in hyaluronic acid with stiffness similar to tumor associated stiffness, culture in isoflurane, culture in MUCl, and culture in cadmium.

Various sources of EPC can be used, the EPC can be either derived from placental sources, cord tissue, and bone marrow. EPCs can also be cultured in vitro to maintain a source of EPCs, or can be induced to produce further differentiated EPCs that can develop into a desired tissue. The cells of the invention can be obtained by mechanically and enzymatically dissociating cells from bone marrow, placental, adipose, or umbilical cord tissue. Mechanical dissociation can be brought about using methods that include, without limitation, chopping and/or mincing the tissue, and/or centrifugation and the like. Enzymatic dissociation of connective tissue and from cell-to-cell associations can be brought about by enzymes including, but not limited to, Blendzyme, DNAse I, collagenase and trypsin, or a cocktail of enzymes found to be effective in liberating cells from the bone marrow sample. The procedure for mechanically and enzymatically isolating a cell of the present invention should not be construed to be limited to the materials and techniques presented herein, but rather it will be recognized that these techniques are well-established and fall well within the scope of experimental optimization performed routinely in the art. In the case of bone marrow-derived EPCs of the invention are isolated from bone marrow. In the isolation of the cells of the invention, bone marrow can be obtained from any animal by any suitable method. A first step in any such method requires the isolation of bone marrow from the source animal. The animal can be alive or dead, so long as cells within bone marrow are viable. Typically, human bone marrow is obtained from a living donor, using well-recognized surgical protocols. The cells of the invention are present in the initially excised or extracted bone marrow, regardless of the method by which bone marrow is obtained. In another embodiment, bone marrow may be obtained from non-human animals. In one embodiment, a bone marrow is removed from the animal. In one embodiment, bone marrow is washed with a physiologically-compatible solution, such as phosphate buffer saline (PBS). The washing step consists of rinsing bone marrow with PBS, agitating the tissue, and allowing the tissue to settle. In one embodiment, bone marrow is dissociated. The dissociation can occur by enzyme degradation and neutralization. Alternatively, or in conjunction with such enzymatic treatment, other dissociation methods can be used such as mechanical agitation, sonic energy, or thermal energy.

In some instances, it may be desirable to further process the dissociated tissue. For example, the dissociated bone marrow can be filtered to isolate cells from other connective tissue. The extracted cells can be concentrated into a pellet. One method to concentrate the cells includes centrifugation, wherein the sample is centrifuged and the pellet retained. The pellet includes the bone marrow-derived EPCs of the invention.

1. A method of generating prophylactic immunity towards neoplastic angiogenesis comprising the steps of:
a) obtaining an endothelial progenitor cell;
b) culturing the endothelial progenitor cell under conditions resembling the tumor microenvironment; and
c) administering products of the cultured endothelial progenitor cells in a manner to stimulate an immune response capable of cross-reacting with tumor associated endothelial cells.

2. The method of claim 1, wherein the endothelial progenitor cell is derived from at least one of placental tissue, adipose tissue, bone marrow, cord blood, menstrual blood, peripheral blood, endothelial cells, umbilical cord, and Wharton's jelly.

3. The method of claim 2, wherein the endothelial progenitor cell is derived from peripheral blood, wherein the peripheral blood is harvested after mobilization of endothelial progenitor cells, wherein the mobilization of the endothelial progenitor cells is accomplished by at least one of administration of G-CSF, administration of flt-3L, and administration of Mozibil.

4. The method of claim 1, wherein the endothelial progenitor cells express at least one ofCD31, VEGFR2, c-kit, and CD34.

5. The method of claim 2, wherein the placental endothelial progenitor cells are extracted by a method selecting for fetal derived endothelial progenitor cells.

6. The method of claim 5, wherein less than 5% of the placental endothelial progenitor cells are of maternal origin.

7. The method of claim 6, wherein selection of fetal placental endothelial progenitor cells is accomplished through a method comprising the steps of:
(i) isolating a mammalian cellular population;
(ii) enriching for a subpopulation of the cells of step (i), which subpopulation expresses a CD⁴⁵− phenotypic profile;
(iii) enriching for a subpopulation of the CD⁴⁵− cells derived from step (ii) which express a CD³⁴+ phenotypic profile; and
(iv) isolating the subpopulation of CD³⁴+ cells derived from step (iii) which express a CD³¹lo/− phenotypic profile, to thereby isolate the endothelial progenitor cells.

8. The method of claim 1, wherein administration of the endothelial progenitor cells or products thereof are administered in an immunogenic manner, wherein the immunogenicity Is endowed by administration in an allogeneic or xenogeneic manner.

9. The method of claim 1, wherein the allogenicity is provided by purposely mismatching donor source of endothelial progenitor cells or products thereof with the recipient, wherein the mismatching is accomplished ensuring a mismatching at least at one allele between donor and recipient.

10. The method of claim 9, wherein the mismatching is performed by HLA mis-matching, wherein the HLA is an HLA allele.

11. The method of claim 10, wherein the HLA allele is one of HLA-A, HLA-B, HLA-C, HLA-DP, HLA-DQ, HLA-DR, and HLA-27.

12. The method of claim 10, wherein the HLA allele is identified by antibodies or genotyping.

13. The method of claim 8, wherein the xenogenicity is provided by one of administration of a xenogeneic endothelial progenitor cell or a product thereof and admixing a xenogeneic cellular component with an allogeneic endothelial progenitor cell or a product thereof.

14. The method of claim 8, wherein the xenogeneic cellular component is one of a nucleic acid, a peptide, a protein, a sugar, and a cellular membrane.

15. The method of claim 8, wherein the allogenicity is endowed by transfection of the cells with an HLA allele.

16. The method of claim 15, wherein the HLA allele is mismatched to the recipient.

17. The method of claim 15, wherein the HLA allele is at least one of a synthetic HLA molecule, xenogeneic, B-7, and B-27.

18. The method of claim 1, wherein augmentation of immunogenicity is performed by culture in interferon gamma.

19. The method of claim 18, wherein the interferon gamma is provided at least one of concentrations and duration sufficient to increase expression of HLA I and HLA II and concentrations and duration sufficient to increase expression of TAP-1.

20. The method of claim 1, wherein the immunogenicity is augmented by culture with an inhibitor of CLIP.
